(19) United States
(12) Patent Application Publication
(10) Pub. No.: US 2026/0232474 A1
(43) Pub. Date: Aug. 13, 2026

(54) ADJUSTABLE FINGER SPLINTS

(71) Applicant: Virak Orthopedic Research LLC, Short Hills, NJ (US)

(72) Inventor: Virak Tan, Short Hills, NJ (US)

(73) Assignee: Virak Orthopedic Research LLC, Short Hills, NJ (US)

(21) Appl. No.: 19/053,331

(22) Filed: Feb. 13, 2025

(51) Int. Cl. A61F 5/01

(52) U.S. Cl. CPC A61F 5/013; A61F 2005/0155; A61F 2005/0172

(57) ABSTRACT

Adjustable finger splints are provided. The finger splints include a base body that is substantially rectangular in shape, wherein the base body comprises a first long side, a second long side, a proximal end, and a distal end, a first arm obliquely extending from the first long side close to the proximal end, and a first tab extending from the second long side close to the proximal end, in which the first tab is shorter than the first arm and the adjustable finger splint is made of a malleable material.

### BACKGROUND OF THE DISCLOSURE

The present disclosure relates to finger splints, specifically finger splints that are made with malleable materials to allow for adjustment based on specific needs.

The mobility of interphalangeal joints and stability of phalanges may be adversely affected by injuries (such as bone fractures, damaged tendon or ligament, joint sprains, and bruises) or diseases (such as osteoarthritis and rheumatoid arthritis). Often time, a splint is needed to immobilize part of the finger to allow proper recovery in orthopedic treatments.

There are many types of finger splints on the market. The known disadvantages of these splints include: (1) some require external or built-in fasteners to hold the splints in place, making it hard for the patient to apply with one hand; (2) some are not adjustable based on the finger size, requiring the manufacturing and inventory of splints of many different sizes; (3) some have very limited adjustability to fit different finger sizes; (4) some do not fit or conform to fingers well; (5) some are too bulky to allow free movement of uninjured joints or other fingers; (6) some cannot be secured on the finger and are easy to fall off; (7) some are made of materials that need to be kept dry all the time, making it difficult to wash hands with the splint on.

There exists a need for improved splints that are adjustable, easy to apply, able to stay in place even without a fastener, and low profile so as not to interfere with the movement of other joints or other fingers.

### SUMMARY

In one embodiment, the present disclosure provides an adjustable finger splint. The adjustable finger splint comprises:
a base body that is substantially rectangular in shape, wherein the base body comprises a first long side, a second long side, a proximal end, and a distal end;
a first arm obliquely extending from the first long side close to the proximal end; and
a first tab extending from the second long side close to the proximal end, wherein the first tab is shorter than the first arm, and
   wherein the adjustable finger splint is made of a malleable material.

In one embodiment, the finger splint is configured to move from either a first plenary position wherein the base body, the first arm, and the first tab are all in a plenary configuration or a first tubular position wherein the first arm and the first tab are curved horizontally to form a tubular shape that can fit loosely around a finger

to a second tubular position wherein the base body abuts a finger to provide support, and the first arm and the first tab are curved around the proximal end of a finger portion to a snug fit around the finger.

In one embodiment, the angle formed by the long axis of the base body and the long axis of the first arm is in the range of 65 to 105 degrees, optionally in the range of 72 to 85 degrees, and optionally in the range of 75 to 83 degrees.

In one embodiment, the angle formed by the long axis of the base body and the long axis of the first tab is in the range of 65 to 105 degrees, optionally in the range of 82 to 95 degrees, and optionally in the range of 85-93 degrees.

In one embodiment, wherein the length of the base body is in the range of 15 to 70 mm, optionally in the range of 25 to 60 mm, and optionally in the range of 25 to 50 mm.

In one embodiment, the width of the base body is in the range of 5 to 20 mm, and optionally in the range of 7 to 15 mm.

In one embodiment, the length of the first arm is in the range of 50 to 130 mm, optionally in the range of 75 to 125 mm, and optionally in the range of 75 to 100 mm.

In one embodiment, the width of the first arm is in the range of 5 to 15 mm, optionally in the range of 7 to 13 mm, and optionally in the range of 7 to 10 mm.

In one embodiment, the first arm is tapered, with its width decreasing from the end adjacent to the base body to the end away from the base body.

In one embodiment, the length of the first tab is in the range of 10 to 40 mm, optionally in the range of 15 to 35 mm, and optionally in the range of 15 to 25 mm.

In one embodiment, the width of the first tab is in the range of 5 to 15 mm, optionally in the range of 7 to 13 mm, and optionally in the range of 7 to 10 mm.

In one embodiment, the edge of the proximal end of the base body is curved inward to form an indent.

In one embodiment, the malleable material is at least one of aluminum, silver, gold, platinum, titanium, palladium, and an alloy thereof. Optionally, the malleable material is aluminum.

In one embodiment, the total thickness of the adjustable finger splint is in the range of 0.8 to 5.0 mm, optionally in the range of 0.8 to 4.0 mm, optionally in the range of 1 to 2 mm, and optionally in the range of 1.0 to 1.5 mm.

In one embodiment, one side of the finger splint is covered with a soft cushion layer. Optionally, the thickness of the soft cushion layer is less than about ¾, optionally less than about ½, of the total thickness of the adjustable finger splint.

In one embodiment, the adjustable finger splint further comprises: a second arm obliquely extending from the first long side close to the distal end; and a second tab extending from the second long side close to the distal end.

In one embodiment, the finger splint is configured to move from either a first plenary position wherein the base body, the first arm, the first tab, the second arm, and the second tab are all in a plenary configuration or a first tubular position wherein the first arm, the first tab, the second arm, and the second tab are curved horizontally to form a tubular shape that can fit loosely around a finger

to a second tubular position wherein the base body abuts a finger to provide support, the first arm and the first tab are curved around the proximal end of a finger portion, and the second arm and the second tab are curved around the distal end of the finger portion to a snug fit around the finger.

In one embodiment, the angle formed by the long axis of the base body and the long axis of the second arm is in the range of 65 to 105 degrees, optionally in the range of 72 to 85 degrees, and optionally in the range of 75 to 83 degrees.

In one embodiment, the angle formed by the long axis of the base body and the long axis of the second tab is in the range of 65 to 105 degrees, optionally in the range of 82 to 95 degrees, and optionally in the range of 85-93 degrees.

In one embodiment, the length of the second arm is in the range of 50 to 125 mm, optionally in the range of 65 to 115 mm, optionally in the range of 65 to 100 mm.

In one embodiment, the width of the second arm is in the range of 5 to 15 mm, optionally in the range of 7 to 13 mm, and optionally in the range of 7 to 10 mm.

In one embodiment, the length of the second tab is in the range of 10 to 40 mm, optionally in the range of 15 to 35 mm, and optionally in the range of 15 to 25 mm

In one embodiment, the width of the second tab is in the range of 5 to 15 mm, optionally in the range of 7 to 13 mm, and optionally in the range of 7 to 10 mm.

In one embodiment, the present disclosure provides an adjustable finger splint. The adjustable finger splint comprises:
a base body in the shape of an elliptical ring or an elliptical ring with a flattened end near the major axis of the elliptical ring;
a first arm extending from the first side of the base body;
a second arm extending from the second side of the base body;
   wherein the first and the second arms are parallel to each other,
   wherein the first and second sides are located on the opposite sides of the major axis of the elliptical ring, and
   wherein the adjustable finger splint is made of a malleable material.

In one embodiment, the finger splint is configured to move from either a first plenary position wherein the base body, the first arm, and the second arm are all in a plenary configuration or a first curved position wherein the first and second sides of the base body and the first and second arms are all curved inward toward the major axis of the elliptical ring to form a ring with an ovoid-shaped base, wherein the ring can fit loosely around a finger

to a second curved position wherein the base body abuts a finger to provide support, and the first and second arms are curved around a finger portion to a snug fit around the finger.

In one embodiment, the length of the major axis of the elliptical ring is in the range of 22 to 45 mm, optionally in the range of 25 to 45 mm.

In one embodiment, the length of the minor axis of the elliptical ring is in the range of 22 to 36 mm, optionally in the range of 25 to 36 mm.

In one embodiment, the length of either arm is in the range of 17 to 35 mm.

In one embodiment, the width of either arm is in the range of 1 to 7 mm, optionally in the range of 2 to 6 mm, optionally in the range of 3 to 5 mm.

In one embodiment, the width of the base body is in the range of 1 to 7 mm, optionally in the range of 2 to 6 mm, optionally in the range of 3 to 5 mm.

In one embodiment, one or both ends near the major axis of the elliptical ring is flattened.

In one embodiment, the flattened end near the major axis of the elliptical ring has a length in the range of 0.6 to 15.0 mm, optionally in the range of 0.8 to 14.0 mm, optionally in the range of 1.0 to 12.0 mm. Optionally, the length of both flattened ends are the same.

In one embodiment, the malleable material is at least one of aluminum, silver, gold, platinum, titanium, palladium, and an alloy thereof. Optionally, the malleable material is aluminum.

In one embodiment, the thickness of the finger splint is in the range of 0.8 to 5.0 mm, optionally in the range of 0.8 to 4.0 mm, optionally in the range of 1 to 2 mm, optionally in the range of 1.0 to 1.5 mm.

In one embodiment, one side of the finger splint is covered with a soft cushion layer. Optionally, the thickness of the soft cushion layer is less than about ¾, optionally less than about ½, of the total thickness of the adjustable finger splint.

### BRIEF DESCRIPTION OF THE DRAWINGS

Embodiments of the present disclosure are hereinafter described in conjunction with the following figures, wherein like numerals denote like elements. The figures are provided for illustration and depict exemplary embodiments of the disclosure. The figures are provided to facilitate understanding of the disclosure without limiting the breadth, scope, scale, or applicability of the disclosure. The drawings are not necessarily made to scale. Details that may be apparent or unnecessary may be omitted to save space or for more effective illustration. Some embodiments may be practiced with additional components or steps and/or without all of the components or steps that are illustrated.

FIG. 1a illustrates an adjustable finger splint with a first arm and a first tab in a plenary configuration. FIG. 1b illustrates a perspective view of the adjustable finger splint in a plenary position. FIGS. 1c and 1d illustrate different perspective views of the adjustable finger splint in a tubular position.

FIG. 2a illustrates an adjustable finger splint with a first arm, a first tab, a second arm, and a second tab in a plenary configuration. FIG. 2b illustrates a perspective view of the adjustable finger splint in a plenary position. FIGS. 2c and 2d illustrate different perspective views of the adjustable finger splint in a tubular position.

FIGS. 3a and 3b each illustrates an adjustable finger splint with a first arm and a second arm in a plenary configuration. FIG. 3c illustrates a perspective top view of the adjustable finger splint in curved position. FIGS. 3d and 3e illustrate different perspective side views of the adjustable finger splint in a curved position.

### DETAILED DESCRIPTION

The following detailed description is exemplary in nature and is not intended to limit the disclosure or the application and uses of the embodiments of the disclosure. Descriptions of specific devices, techniques, and applications are provided only as examples. Modifications to the examples described herein will be readily apparent to those of ordinary skill in the art, and the general principles defined herein may be applied to other examples and applications without departing from the spirit and scope of the disclosure. The present disclosure should be accorded scope consistent with the claims, and not limited to the examples described and shown herein.

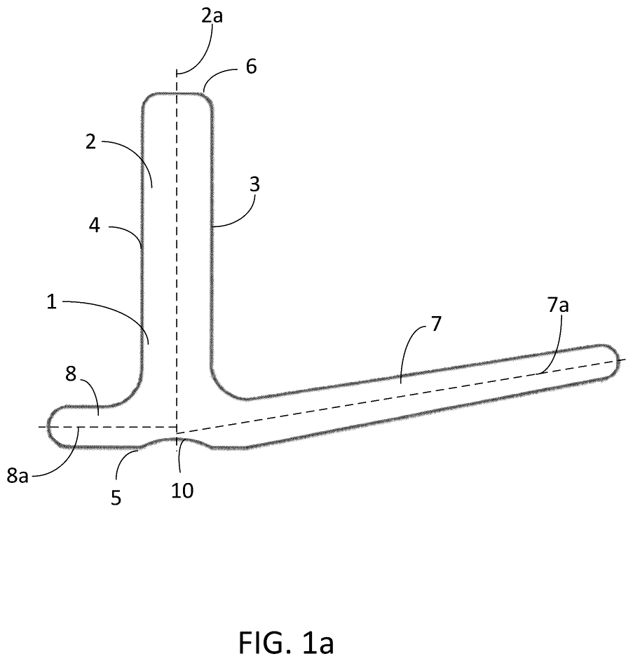

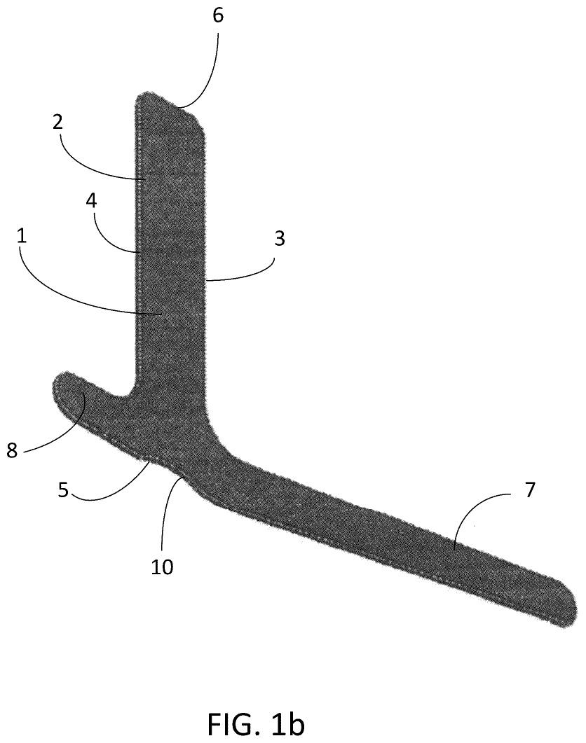

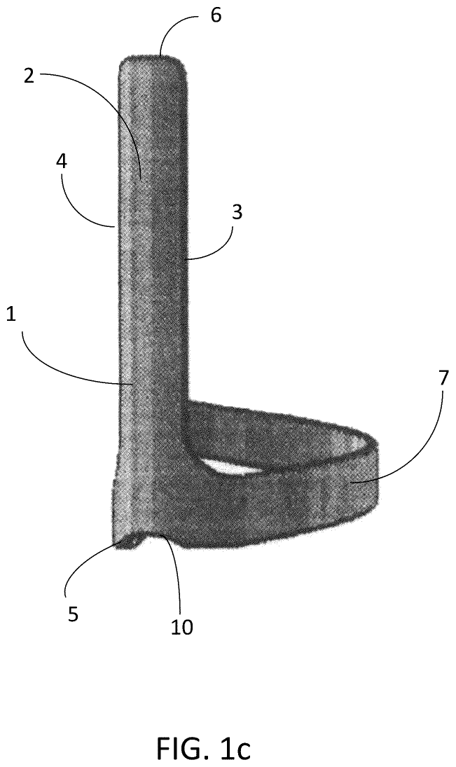

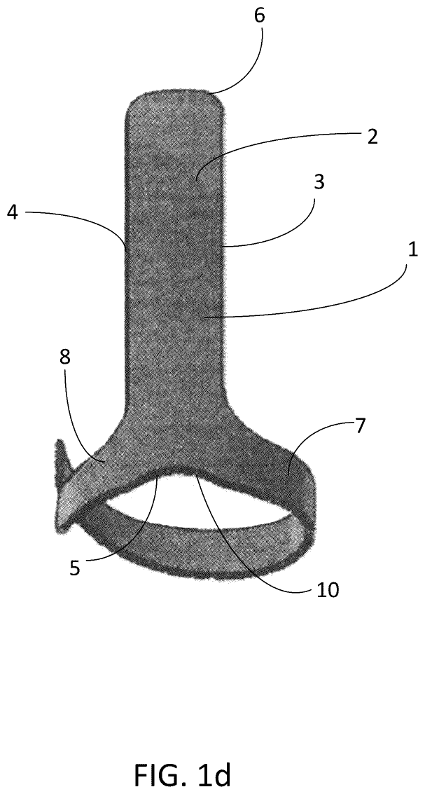

FIGS. 1a-1d show an embodiment of the present disclosure: An adjustable finger splint (1) comprising a base body (2). The base body (2) is substantially rectangular in shape. It comprises a first long side (3), a second long side (4), a proximal end (5), and a distal end (6).

The finger splint (1) also comprises a first arm (7). The first arm (7) obliquely extends from the first long side (3) close to the proximal end (5).

The finger splint (1) also comprises a first tab (8). The first tab (8) extends from the second long side (4) close to the proximal end (5).

Optionally, the first tab (8) is shorter than the first arm (7).

The finger splint (1) is configured to move from a first position to a second tubular position. The first position can be a first plenary position as illustrated in FIGS. 1a and 1b, or a first tubular position as illustrated in FIGS. 1c and 1d. The second tubular position is also illustrated in FIGS. 1c and 1d.

When the finger splint (1) is in a first plenary position, the base body (2), the first arm (7), and the first tab (8) are all in a plenary configuration. Optionally, the finger splint (1) can be packaged, shipped, stored, and sold in this position.

When the finger splint (1) is in use, it is preferably in a second tubular position, wrapping around a finger, supporting a portion of the finger that requires protection. In this position, the base body (2) abuts a finger, providing support.

The splint can be placed anywhere along the finger. The base body can be in any orientation around the long axis of the finger, i.e., dorsal, volar, etc. The tubular (i.e., “ring”) portion can be proximal or distal. Optionally, the base body can be bent in any direction to best fit the finger it supports. For example, either the first long side (3) or the second long side (4) can be bent in a direction perpendicular to the long axis (2a) of the base body (2) to curve toward the finger. The base body (2) can also be bent in the direction of the long axis (9) to the desired angle of flexion.

Optionally, both the first arm (7) and the first tab (8) are bent in a horizontal direction (perpendicular to the long axis (2a)) and curved along the circumference of the finger toward the proximal end to provide a snug fit. The first arm and first tab serve to create an open “ring” that fits around the finger, making it adjustable for different finger sizes (circumferences). The obliqueness of the first arm can avoid overlap, impingement, or inference with the first tab when curved along the circumference of the finger.

When each part of the finger splint (1) is bent properly and placed in the proper position, the finger splint (1) can stay on the finger to provide support and immobilization without any additional fastener to hold it in place. Optionally, additional fasteners such as tapes, rubber bands, and adhesives can be used as a supplement.

Optionally, the finger splint (1) can be pre-formed into a first tubular position prior to being applied to a finger. The first tubular position is similar to the second tubular position, except that the base body (2), the first arm (7), and the first tab (8) are bent into a non-final shape that would allow a finger to be inserted. Specifically, the first arm (7) and the first tab (8) are bent in a horizontal direction to form a tubular shape that can fit loosely around a finger. Optionally, the finger splint (1) can be packaged, shipped, stored, and sold in this tubular position. With pre-formed finger splints, only minor adjustments are needed in application.

The finger splint (1) is preferably made of a malleable material. The malleable material allows the base body (2), the first arm (7), and the first tab (8) to be bent and the finger splint (1) to move from a plenary position to a tubular position. The malleable material can be a metal or synthetic material. Optionally, it is at least one of aluminum, silver, gold, platinum, titanium, palladium, and an alloy thereof. Optionally, it is aluminum.

Optionally, the malleable material is soft enough to be cut and trimmed with a pair of trauma shears.

Optionally, the malleable material is water-resistant to allow washing of the finger with the finger splint on.

Optionally, the finger splint (1) has a low profile, and does not interfere with the movement of other fingers or joints. Optionally, the total thickness of the finger splint (1) is in the range of 0.8 to 5.0 mm, optionally in the range of 0.8 to 4 mm, optionally in the range of 1 to 2 mm, and optionally in the range of 1.0 to 1.5 mm.

An additional soft cushion layer can be applied to the finger splint (1) on the side that faces the finger. Optionally, the soft cushion layer is water-resistant to allow washing of the finger with the finger splint on. Optionally, the thickness of the soft cushion layer is less than about ¾, optionally less than about ½, of the total thickness of the adjustable finger splint.

Optionally, the angle formed by the long axis (2a) of the base body (2) and the long axis (7a) of the first arm (7) is in the range of 65 to 105 degrees, optionally in the range of 72 to 85 degrees, and optionally in the range of 75 to 83 degrees. The angle can avoid overlap, impingement, or inference with the first tab when the first arm is curved along the circumference of the finger.

Optionally, the angle formed by the long axis (2a) of the base body (2) and the long axis (8a) of the first tab (8) is in the range of 65 to 105 degrees, optionally in the range of 82 to 95 degrees, and optionally in the range of 85 to 93 degrees. The angle can avoid overlap, impingement or inference with the first arm when the first tab is curved along the circumference of the finger.

The length of the base body (2) can be chosen based on the length of the finger portion to be protected and the specific support function it serves. Optionally, the length is in the range of 15 to 70 mm, optionally in the range of 25 to 60 mm, and optionally in the range of 25 to 50 mm.

The width of the base body (2) can be chosen based on the size of the finger portion to be protected and the specific support function it serves. Optionally, the width is in the range of 5 to 20 mm, and optionally in the range of 7 to 15 mm.

The length of the first arm (7) can be chosen based on the size of the finger portion to be protected and the specific support function it serves. Optionally, the length is in the range of 50 to 130 mm, optionally in the range of 75 to 125 mm, and optionally in the range of 75 to 100 mm.

The width of the first arm (7) can be chosen based on the size of the finger portion to be protected and the specific support function it serves. Optionally, the width is in the range of 5 to 15 mm, optionally in the range of 7 to 13 mm, and optionally in the range of 7 to 10 mm.

The width of the first arm (7) does not need to stay constant from one end to the other. Optionally, the first arm is tapered, with its width decreasing from the end adjacent to the base body to the end away from the base body.

The length of the first tab (8) can be chosen based on the size of the finger portion to be protected and the specific function it serves. Optionally, the length is in the range of 10 to 40 mm, optionally in the range of 15 to 35 mm, and optionally in the range of 15 to 25 mm.

The width of the first tab (8) can be chosen based on the size of the finger portion to be protected and the specific function it serves. Optionally, the width is in the range of 5 to 15 mm, optionally in the range of 7 to 13 mm, and optionally in the range of 7 to 10 mm.

Optionally, the edge of the proximal end (5) of the base body (2) is curved inward to form an indent (10). The indent provides clearance for finger flexion when the splint is applied to the volar (face) side of the finger.

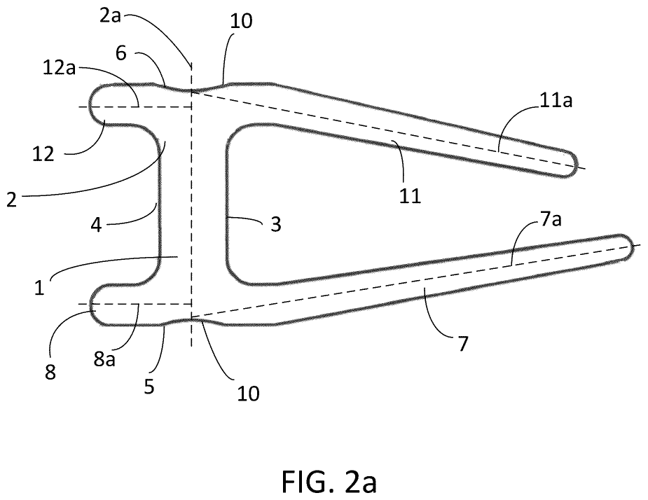

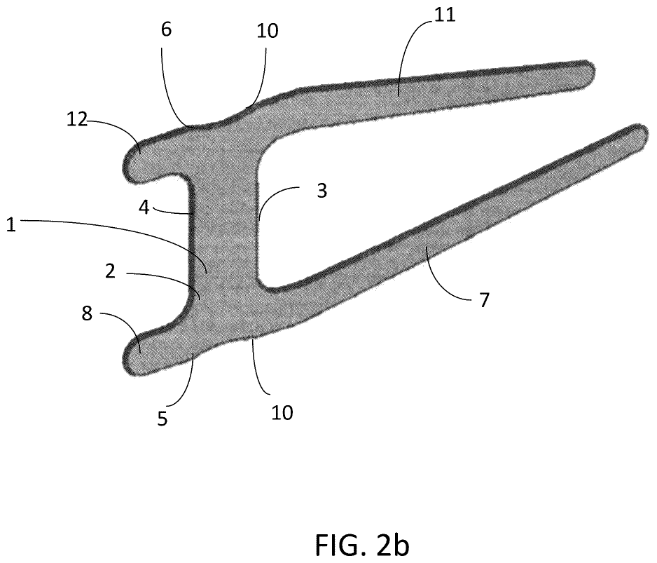

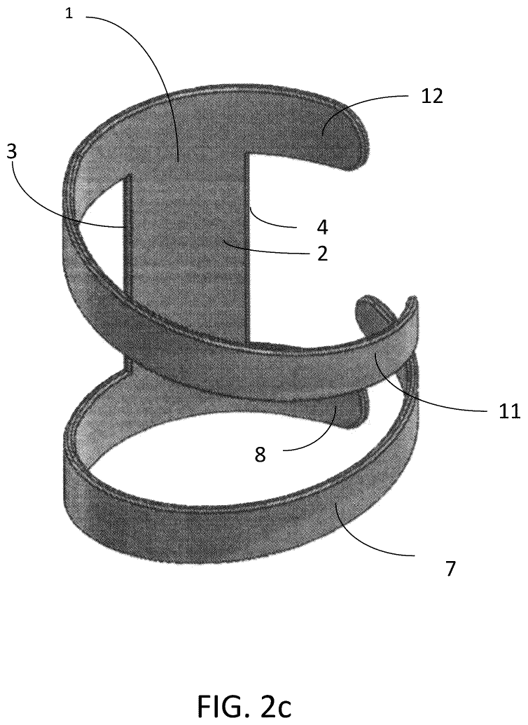

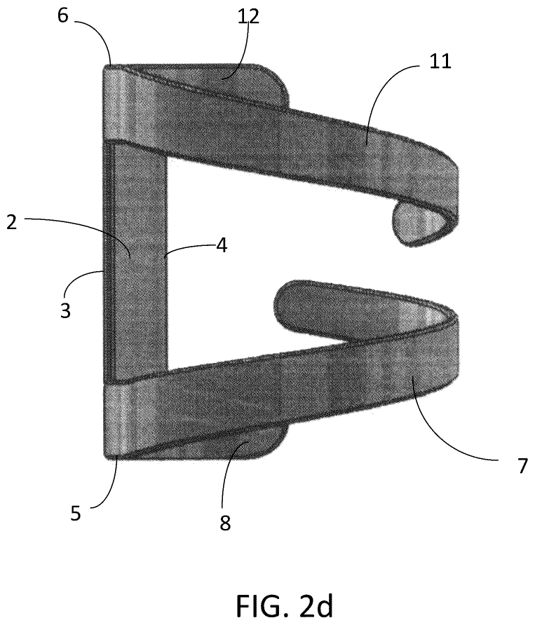

FIGS. 2a-2d show an embodiment of the present disclosure: An adjustable finger splint (1) comprising a base body (2). Similar to the embodiment illustrated in FIGS. 1a-1d, the base body (2) is substantially rectangular in shape. The finger splint (1) comprises a first long side (3), a second long side (4), a proximal end (5), and a distal end (6). It comprises a first arm (7) that obliquely extends from the first long side (3) close to the proximal end (5) and a first tab (8) that extends from the second long side (4) close to the proximal end (5).

In addition, the finger splint (1) comprises a second arm (11). The second arm (11) obliquely extends from the first long side (3) close to the distal end (6).

The finger splint (1) comprises a second tab (12). The second arm (12) extends from the second long side (4) close to the distal end (6).

Optionally, the second tab (12) is shorter than the second arm (11).

The finger splint (1) is configured to move from a first position to a second tubular position. The first position can be a first plenary position as illustrated in FIGS. 2a and 2b, or a first tubular position as illustrated in FIGS. 2c and 2d. The second tubular position is also illustrated in FIGS. 2c and 2d.

When the finger splint (1) is in a first plenary position, the base body (2), the first arm (7), the first tab (8), the second arm (11), and the second tab (12) are all in a plenary configuration.

When the finger splint (1) is in use, it is preferably in a second tubular position, wrapping around a finger, supporting a portion of the finger that requires protection. In this position, the base body (2) abuts a finger, providing support. Optionally, the base body can be bent in any direction to best fit the finger it supports. For example, either the first long side (3) or the second long side (4) can be bent in a direction perpendicular to the long axis (2a) of the base body (2) to curve toward the finger. The base body (2) can also be bent in the direction of the long axis (9) to the desired angle of flexion.

Optionally, the first arm (7) and the first tab (8) are bent in a horizontal direction and curved along the circumference of the finger toward the proximal end, and the second arm (11) and the second tab (12) are bent in a horizontal direction and curved along the circumference of the finger toward the distal end to provide a snug fit. The combination of the first arm and first tab and the combination of the second arm and the second tab each serves to create an open “ring” that fits around the finger, making it adjustable for different finger sizes (circumferences). The obliqueness of the first and the second arm can avoid overlap, impingement, or inference with the first tab and second tab, respectively, when curved along the circumference of the finger.

When each part of the finger splint (1) is bent properly and placed in the proper position, the finger splint (1) can stay on the finger to provide support and immobilization without any additional fastener to hold it in place. Optionally, additional fasteners such as tapes, rubber bands, adhesives can be used as a supplement.

Optionally, the finger splint (1) can be pre-formed into a first tubular position prior to being applied to a finger. The first tubular position is similar to the second tubular position, except that the base body (2), the first arm (7), the second arm (11), the first tab (8), and the second tab (12) are bent into a non-final shape that would allow a finger to be inserted. Specifically, the first arm (7) and the first tab (8) are bent in a horizontal direction to form a tubular shape that can fit loosely around a finger. Similarly, the second arm (11) and the second tab (12) are also bent in a horizontal direction to form a tubular shape that can fit loosely around a finger. Optionally, the finger splint (1) can be packaged, shipped, stored, and sold in this tubular position. With pre-formed finger splints, only minor adjustments are needed in application.

The finger splint (1) is preferably made of a malleable material. The malleable material allows the base body (2), the first arm (7), the second arm (11), the first tab (8), and the second tab (12) to be bent and the finger splint (1) to move from a plenary position to a tubular position. The malleable material can be a metal or synthetic material. Optionally, it is at least one of aluminum, silver, gold, platinum, titanium, palladium, and an alloy thereof. Optionally, it is aluminum.

Optionally, the malleable material is soft enough to be cut and trimmed with a pair of trauma shears.

Optionally, the malleable material is water-resistant to allow washing of the finger with the finger splint on.

Optionally, the finger splint (1) has a low profile, and does not interfere with the movement of other fingers or joints. Optionally, the total thickness of the finger splint (1) is in the range of 0.8 to 5.0 mm, optionally in the range of 0.8 to 4 mm, optionally in the range of 1 to 2 mm, and optionally in the range of 0.8 to 1.5 mm.

An additional soft cushion layer can be applied to the finger splint (1) on the side that faces the finger. Optionally, the soft cushion layer is water-resistant to allow washing of the finger with the finger splint on. Optionally, the thickness of the soft cushion layer is less than about ¾, optionally less than about ½, of the total thickness of the adjustable finger splint.

Optionally, the angle formed by the long axis (2a) of the base body (2) and the long axis (7a) of the first arm (7) or the angle formed by the long axis (2a) of the base body (2) and the long axis (11a) of the second arm (11) is in the range of 65 to 105 degrees, optionally in the range of 72 to 85 degrees, and optionally in the range of 75 to 83 degrees. The angle can avoid overlap, impingement, or inference with the first tab when the first arm or the second arm is curved along the circumference of the finger.

Optionally, the angle formed by the long axis (2a) of the base body (2) and the long axis (8a) of the first tab (8) or the angle formed by the long axis (9) of the base body (2) and the long axis (12a) of the second tab (12) is in the range of 65 to 105 degrees, optionally in the range of 82 to 95 degrees, and optionally in the range of 85 to 93 degrees. The angle can avoid overlap, impingement or inference with the first arm when the first tab or the second tab is curved along the circumference of the finger.

The length of the base body (2) can be chosen based on the length of the finger portion to be protected and the specific support function it serves. Optionally, the length is in the range of 15 to 70 mm, optionally in the range of 25 to 60 mm, and optionally in the range of 25 to 50 mm.

The width of the base body (2) can be chosen based on the size of the finger portion to be protected and the specific support function it serves. Optionally, the width is in the range of 5 to 20 mm, and optionally in the range of 7 to 15 mm.

The length of the first arm (7) can be chosen based on the size of the finger portion to be protected and the specific support function it serves. Optionally, the length is in the range of 50 to 130 mm, optionally in the range of 75 to 125 mm, and optionally in the range of 75 to 100 mm.

The length of the second arm (11) can be chosen based on the size of the finger portion to be protected and the specific support function it serves. Optionally, the length is in the range of 50 to 125 mm, optionally in the range of 65 to 115 mm, optionally in the range of 65 to 100 mm. The second arm is optionally shorter than the first arm because at the distal end (closer to the tip) of the finger typically has a smaller circumference.

The width of the first arm (7) and of the second arm (11) can be chosen based on the size of the finger portion to be protected and the specific support function it serves. Optionally, the width is in the range of 5 to 15 mm, optionally in the range of 7 to 13 mm, and optionally in the range of 7 to 10 mm.

The width of the first arm (7) or of the second arm (11) does not need to stay constant from one end to the other. Optionally, the first arm or the second arm is tapered, with its width decreasing from the end adjacent to the base body to the end away from the base body.

The length of the first tab (8) and of the second tab (12) can be chosen based on the size of the finger portion to be protected and the specific function it serves. Optionally, the length is in the range of 10 to 40 mm, optionally in the range of 15 to 35 mm, and optionally in the range of 15 to 25 mm.

The width of the first tab (8) and of the second tab (12) can be chosen based on the size of the finger portion to be protected and the specific function it serves. Optionally, the width is in the range of 5 to 15 mm, optionally in the range of 7 to 13 mm, and optionally in the range of 7 to 10 mm.

Optionally, the edge of the proximal end (5) or the edge of the distal end (6) of the base body (2) is curved inward to form an indent (10). The indent provides clearance for finger flexion when the splint is applied to the volar (face) side of the finger.

The first arm and first tab serve to create an open “proximal ring” that fits around the proximal end of the finger. The second arm and second tab serve to create an open “distal ring” that fits around the distal end of the finger. The rings make the splint adjustable for different finger sizes (circumferences). The proximal ring is conceived to be proximal to and the distal ring distal to the region of the finger requiring protection. The obliqueness of the arms can avoid overlap, impingement, or inference with the tabs when curved along the circumference of the finger.

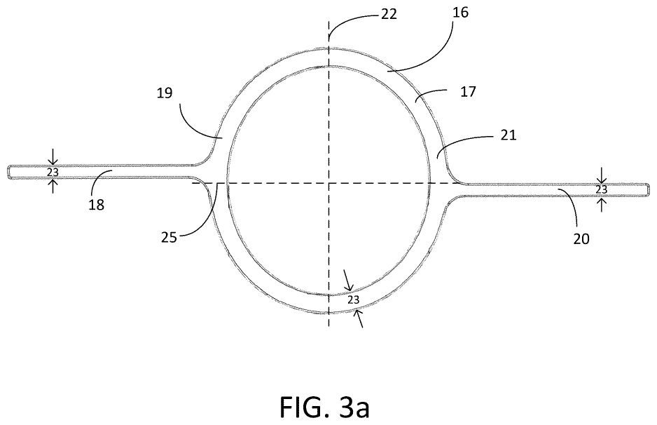

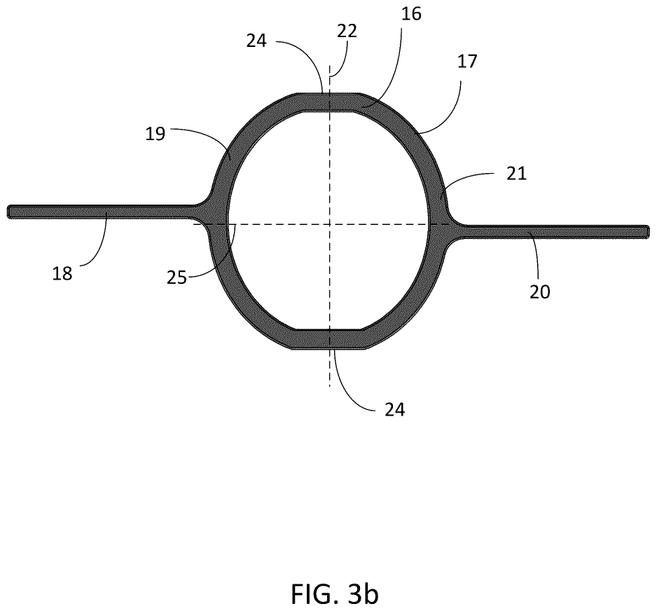

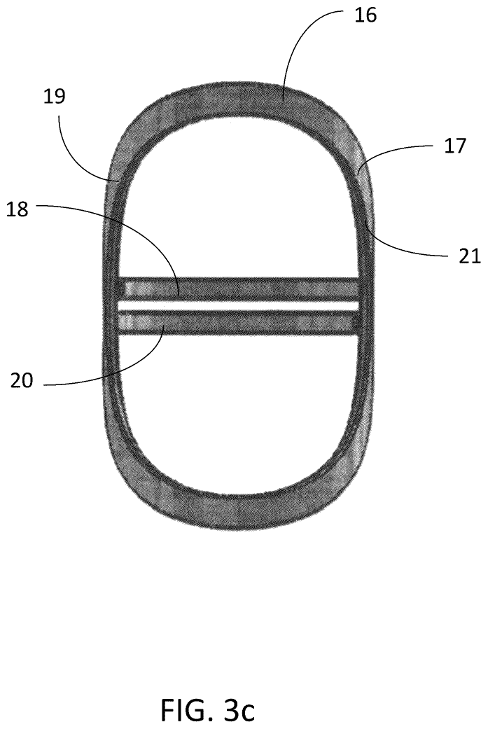

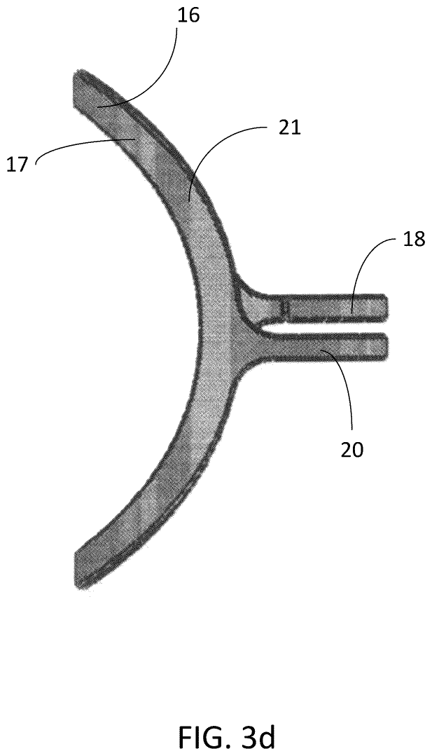

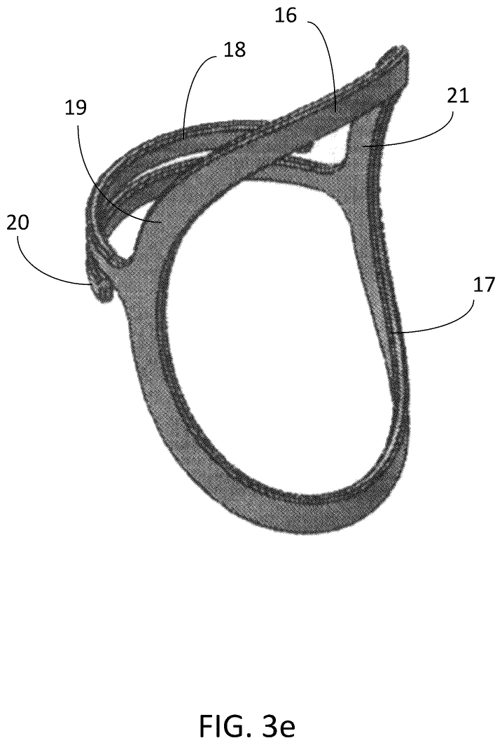

FIGS. 3a-3e show another embodiment of the present disclosure: An adjustable finger splint (16) comprising a base body (17). The base body (17) is in the shape of an elliptical ring in FIG. 3a, or is in the shape of an elliptical ring with flattened ends near the major axis (22) of the elliptical ring in FIG. 3b.

The finger splint (16) also comprises a first arm (18). The first arm (18) extends from the first side (19) of the base body (17).

The finger splint (16) also comprises a second arm (20). The first arm (20) extends from the first side (21) of the base body (17). The first arm (18) and the second arm (20) are parallel to each other and located on the opposite sides of the major axis (22) of the elliptical ring.

The finger splint (16) is configured to move from a first position to a second curved position. The first position can be a plenary position as illustrated in FIGS. 3a and 3b, or a curved position as illustrated in FIGS. 3c-3e. The second curved position is also illustrated in FIGS. 3c-3e.

When the finger splint (16) is in a first plenary position, the base body (17), the first arm (18), and the second arm (20) are all in a plenary configuration. Optionally, the finger splint (16) can be packaged, shipped, stored, and sold in this position.

When the finger splint (16) is in use, it is preferably in a second curved position, wrapping around a finger, supporting a portion of the finger that requires protection. In this position, the base body (17) abuts a finger, providing support. The finger splint (16) can be used as an extension block for the interphalangeal joint by placing the first arm (18) and the second arm (20) at the joint flexion crease. When the arms are not aligned with the joint flexion crease, the splint is static preventing movement of the interphalangeal joint.

Optionally, the base body (17) can be bent in any direction to best fit the finger it supports. For example, the first side (19) and second side (21) of the base body can be bent in a horizontal direction (perpendicular to the major axis (22) of the elliptical ring) to form an ovoid-shaped base. The first arm (18) and the second arm (20) can also be bent in a horizontal direction toward each other to form two rings on the ovoid-shaped base. Preferably, the the first arm (18) and the second arm (20) do not touch each other. The two rings on the ovoid-shaped base can be adjusted to form a snug fit on the finger to be protected. Any free hanging end of the first arm (18) or the second arm (20) can be trimmed away.

When each part of the finger splint (16) is bent properly and placed in the proper position, the finger splint (16) can stay on the finger to provide support and immobilization without any additional fastener to hold it in place. Optionally, additional fasteners such as tapes, rubber bands, adhesives can be used as a supplement.

Optionally, the finger splint (16) can be pre-formed into a first curved position prior to being applied to a finger. The first curved position is similar to the second curved position, except that the base body (17), the first arm (18), and the second arm (20) are bent into a non-final shape that would allow a finger to be inserted. Specifically, the first side (19) and second side (21) of the base body can be bent in a horizontal direction to form an ovoid-shaped base, and the first arm (18) and the second arm (20) can be bent in a horizontal direction toward each other to form two rings on the ovoid-shaped base. Optionally, the finger splint (16) can be packaged, shipped, stored, and sold in this tubular position. With pre-formed finger splints, only minor adjustments are needed in application.

The finger splint (16) is preferably made of a malleable material. The malleable material allows the base body (17), the first arm (18), and the second arm (20) to be bent and the finger splint (16) to move from a plenary position to a curved position. The malleable material can be a metal or synthetic material. Optionally, it is at least one of aluminum, silver, gold, platinum, titanium, palladium, and an alloy thereof. Optionally, it is aluminum.

Optionally, the malleable material is soft enough to be cut and trimmed with a pair of trauma shears.

Optionally, the malleable material is water-resistant to allow washing of the finger with the finger splint on.

Optionally, the finger splint (16) has a low profile, and does not interfere with the movement of other fingers or joints. Optionally, the total thickness of the finger splint (16) is in the range of 0.8 to 5.0 mm, optionally in the range of 0.8 to 4 mm, optionally in the range of 1 to 2 mm, and optionally in the range of 0.8 to 1.5 mm.

An additional soft cushion layer can be applied to the finger splint (16) on the side that faces the finger. Optionally, the soft cushion layer is water-resistant to allow washing of the finger with the finger splint on. Optionally, the thickness of the soft cushion layer is less than ¾, optionally less than about ½, of the total thickness of the adjustable finger splint.

Optionally, the length of the major axis (22) of the base body (17) is in the range of 22 to 45 mm, optionally in the range of 25 to 45 mm.

Optionally, one or both ends (24) near the major axis of the elliptical ring is flattened, as illustrated in FIG. 3(b).

Optionally, the length of the flattened end (24) is in the range of 0.6 to 15.0 mm, optionally in the range of 0.8 to 14.0 mm, optionally in the range of 1.0 to 12.0 mm. Optionally, the length of both flattened ends are the same.

Optionally, the length of the minor axis (25) of the base body (17) is in the range of 22 to 36 mm, optionally in the range of 25 to 36 mm.

Optionally, the length of the first arm (18) or the second arm (20) is in the range of 17 to 35 mm.

Optionally, the width (23) of the base body (17), the first arm (18), or the second arm (20) is in the range of 1 to 7 mm, optionally in the range of 2 to 6 mm, optionally in the range of 3 to 5 mm.

The splints of the present disclosure have the advantages of being adjustable, easy to apply, able to stay in place even without a fastener, and low profile so as not to interfere with the movement of other joints or other fingers. Use of these splints can be 24/7 without having to remove the splint for hand washing, showering, and personal hygiene.

What is claimed is:

1. An adjustable finger splint, comprising:
a base body that is substantially rectangular in shape, wherein the base body comprises a first long side, a second long side, a proximal end, and a distal end;
a first arm obliquely extending from the first long side close to the proximal end; and
a first tab extending from the second long side close to the proximal end, wherein the first tab is shorter than the first arm, and
wherein the adjustable finger splint is made of a malleable material.

2. The adjustable finger splint of claim 1, wherein the finger splint is
configured to move
from either a first plenary position wherein the base body, the first arm, and the first tab are all in a plenary configuration or a first tubular position wherein the first arm and the first tab are curved horizontally to form a tubular shape that can fit loosely around a finger
to a second tubular position wherein the base body abuts a finger to provide support, and the first arm and the first tab are curved around the proximal end of a finger portion to a snug fit around the finger.

3. The adjustable finger splint of claim 1, wherein the angle formed by the long axis of the base body and the long axis of the first arm is in a range of 72 to 85 degrees, and the angle formed by the long axis of the base body and the long axis of the first tab is in a range of 82 to 95 degrees.

4. The adjustable finger splint of claim 1, wherein the length of the base body is in a range of 25 to 60 mm and the width of the base body is in a range of 7 to 15 mm.

5. The adjustable finger splint of claim 1, wherein the length of the first arm is in a range of 75 to 125 mm and the width of the first arm is in a range of 7 to 13 mm.

6. The adjustable finger splint of claim 1, wherein the first arm is tapered such that its width decreases from the end adjacent to the base body to the end away from the base body.

7. The adjustable finger splint of claim 1, wherein the length of the first tab is in a range of 15 to 35 mm and the width of the first tab is in a range of 7 to 13 mm.

8. The adjustable finger splint of claim 1, wherein the edge of the proximal end of the base body is curved inward to form an indent.

9. The adjustable finger splint of claim 1, wherein the malleable material is at least one of aluminum, silver, gold, platinum, titanium, palladium, and an alloy thereof.

10. The adjustable finger splint of claim 1, wherein the total thickness of the adjustable finger splint is in a range of 1 to 2 mm.

11. The adjustable finger splint of claim 1, wherein one side of finger splint is covered with a soft cushion layer, wherein the ratio of the thickness of the soft cushion layer is less than ¾ of the total thickness of the adjustable finger splint.

12. The adjustable finger splint of claim 1, further comprising:
a second arm obliquely extending from the first long side close to the distal end; and
a second tab extending from the second long side close to the distal end.

13. The adjustable finger splint of claim 12, wherein the finger splint is configured to move
from either a first plenary position wherein the base body, the first arm, the first tab, the second arm, and the second tab are all in a plenary configuration or a first tubular position wherein the first arm, the first tab, the second arm, and the second tab are curved horizontally to form a tubular shape that can fit loosely around a finger
to a second tubular position wherein the base body abuts a finger to provide support, the first arm and the first tab are curved around the proximal end of a finger portion, and the second arm and the second tab are curved around the distal end of the finger portion to a snug fit around the finger.

14. The adjustable finger splint of claim 12, wherein the angle formed by the long axis of the base body and the long axis of the second arm is in the range of 72 to 85 degrees, and the angle formed by the long axis of the base body and the long axis of the second tab is in the range of 82 to 95 degrees.

15. The adjustable finger splint of claim 12, wherein the length of the second arm is about in the range of 65 to 115 mm and the width of the second t is in the range of 7 to 13 mm.

16. An adjustable finger splint comprising:
a base body in the shape of an elliptical ring or an elliptical ring with a flattened end near the major axis of the elliptical ring;
a first arm extending from the first side of the base body;
a second arm extending from the second side of the base body;
wherein the first and the second arms when hypothetically extended are parallel at a distance to each other,
wherein the first and second sides are located on the opposite sides of the major axis of the elliptical ring, and
wherein the adjustable finger splint is made of a malleable material.

17. The adjustable finger splint of claim 16, wherein the length of the major axis of the elliptical ring is in the range of 22 to 45 mm and the length of the minor axis of the elliptical ring is in the range of 25 to 36 mm.

18. The adjustable finger splint of claim 16, wherein the length of either arm is in the range of 17 to 35 mm and the width of the base body or either arm is in the range of 2 to 6 mm.

19. The adjustable finger splint of claim 16, wherein the length of the flattened end is in the range of 0.8 to 14.0 mm.

20. The adjustable finger splint of claim 16, wherein the finger splint is configured to move
from either a first plenary position wherein the base body, the first arm, and the second arm are all in a plenary configuration or a first curved position wherein the first and second sides of the base body and the first and second arms are all curved inward toward the major axis of the elliptical ring to form a ring with an ovoid-shaped base, wherein the ring can fit loosely around a finger
to a second curved position wherein the base body abuts a finger to provide support, and the first and second arms are curved around a finger portion to a snug fit around the finger.
